(19) United States
(12) Patent Application Publication
(10) Pub. No.: US 2007/0299363 A1
(43) Pub. Date: Dec. 27, 2007

(54) Fecalator and method for concentrating parasite eggs and/or larvae

(76) Inventor: Johnson N. S. Wong, Rolling Hills, CA (US)

Correspondence Address: Robert Berliner;BERLINER & ASSOCIATES, 31st Floor, 555 W. Fifth Street, Los Angeles, CA, 90013, US

(21) Appl. No.: 11/448,330

(22) Filed: Jun. 7, 2006

(51) Int. Cl. A61B 10/00

(52) U.S. Cl. 600/562

(57) ABSTRACT

An apparatus and method for concentrating parasitic eggs and/or larvae from a fecal sample using a single tube by extruding the fecal sample through a strainer using a cap plunger device into a centrifuge tube containing 10% buffered formalin or equivalent fixative. After mixing and fixation, the strainer device is removed, and ethyl acetate and a surfactant are added. After mixing, the extruded sample is centrifuged to concentrate parasite eggs and/or larvae at the bottom of the centrifuge tube.

### FIELD OF THE INVENTION

The invention relates to an apparatus and method for the recovery of parasitic eggs and larvae from feces.

### BACKGROUND OF THE INVENTION

The recovery of parasitic eggs and larvae from feces is of major importance in the diagnosis of many diseases. Early methods for the recovery of parasitic eggs and larvae from feces were impractical for the routine parasitology laboratory. The formalin-ether method, as disclosed in Laboratory Procedures for the Diagnosis of Intestinal Parasites, 1974, U.S. Department of HEW, PHS Pub. No. 1969, pages 103-105, was time consuming and esthetically distasteful. An improved apparatus and method was developed in the 1970's, described and claimed in U.S. Pat. No. 4,081,356 to Zierdt and assigned to the United States as represented by the Department of Health, Education and Welfare. The teachings of U.S. Pat. No. 4,081,356 are incorporated herein by reference. It enables the recovery of parasitic eggs and larvae from a small feces sample in a clean, efficient and quick manner.

A drawback of the Zierdt method is the need to use numerous steps and components as well as a specially constructed centrifuge tube. The underlying methodology is to shake a solvent emulsified sample of feces through a filter into a centrifuge tube, but to accomplish that an inordinate amount of closely manufactured components is used. A feces sample cup with a handle must be manufactured to closely fit to one side of an emulsification chamber so as to be leak proof, the opposite side of the emulsification chamber being threadably fitted to a connector assembly that links the emulsification chamber to a sample receiving separation chamber. The separation chamber serves as a centrifuge tube but must be specially manufactured to have screw threads at its open end. The connecting assembly itself is complex. It is a hollow cylinder formed with screw threads on opposite sides to engage the emulsification chamber on one side and the centrifuge tube on the other side. A stainless steel filter disk fitted with a gas pressure relief tube is supported by a collar formed within the connector assembly. These numerous close fitting components result in a relatively high cost.

### BRIEF SUMMARY OF THE INVENTION

The present invention overcomes the foregoing drawbacks. In contrast to the above-described Zierdt method, where a solvent emulsified sample of feces is shaken through a filter into a specially manufactured centrifuge tube, the present invention uses a plunger to extrude the feces sample through a screen into a standard 15 ml polypropylene centrifuge tube containing 10% buffered formalin. The result is a relatively inexpensive apparatus and simplified method that many small laboratories equipped with a table top centrifuge can benefit from.

More particularly, in one embodiment, a method is provided for concentrating parasitic eggs and/or larvae from a fecal sample, comprising extruding a fecal sample through a strainer into a centrifuge tube containing 10% buffered formalin or another fixative such as sodium acetate formalin (SAF) or zinc polyvinyl alcohol (PVA). After shaking, the strainer is removed and ethyl acetate and Triton X-100 are added to the centrifuge tube. The centrifuge tube containing the extruded sample, the fixative, ethyl acetate and surfactant is shaken and then centrifuged to concentrate parasite eggs and/or larvae at the bottom of the tube.

In another embodiment, an apparatus is provided for extruding a fecal sample into a centrifuge tube and recovering parasitic eggs and/or larvae by centrifugation, comprising a hollow sample cup for receiving the fecal sample having an outer dimension enabling it to fit within the open end of the centrifuge tube. The sample cup has inner and outer walls, an open top end and a bottom end formed with a strainer for the fecal sample. A plunger has a stem normal to having a bottom end that close fits the inner wall of the sample cup and is used to extrude the fecal sample through the strainer. A press assembly for the plunger is formed with a tubular member fitting over, and coterminous with the plunger stem at its bottommost position, constructed so as to bear down on the bottom end of the plunger. A hollow thumb cap is carried on the upper end of the tubular member, open at its bottom end and formed with a lid having upper and lower surfaces, the tubular member being axially secured normal to the lower surface of the lid. The upper surface of the lid has a size to accommodate a user's thumb, facilitating extrusion of the fecal sample. The top end of the sample cup is formed with a flared edge region sized to close fit inside the hollow cap extends to close the centrifuge tube when the plunger extends fully into the sample cup.

The thumb cap is formed with internal screw threads and the open end of the centrifuge tube is formed with mating screw threads, to enable an assembly of the plunger, sample cup and centrifuge tube to be sealed together so that the assembly can be shaken to thoroughly mix the components and homogenize the mixture.

The fecal sample is extruded by placing it in the strainer-fitted sample cup and inserted the sample cup into the centrifuge tube. The plunger is inserted and, aided by the press assembly, is pushed into the sample cup to extrude the fecal sample through the strainer and into the centrifuge tube. After mixing, the sample cup with its plunger is removed. After adding ethyl acetate and Triton X-100, the pressure assembly cap is reapplied to the centrifuge tube. After shaking, the extruded sample is then centrifuged to concentrate parasite eggs and/or larvae at the bottom of the centrifuge tube.

### BRIEF DESCRIPTION OF THE DRAWINGS

For a more complete understanding of the present invention, reference is now made to the following descriptions taken in conjunction with the accompanying drawing, in which:

FIG. 1 is a perspective, exploded view of the apparatus of this invention;

FIG. 2 is a cross-sectional view of the plunger component of the apparatus, taken on line 2-2 of FIG. 1;

FIG. 3 is a cross-sectional view of the apparatus;

FIG. 4 is an enlarged detail of the plunger and pressure cap components of the apparatus;

FIG. 5 is a cross-sectional view of the sample cup component of the apparatus;

FIG. 6 is a cross-sectional view of the sample cup taken on line 6-6 of FIG. 5, showing a plan view of the strainer component of the apparatus; and

FIG. 7 is a perspective view of a centrifuge tube after extrusion into it of fecal matter and centrifugation.

### DETAILED DESCRIPTION OF THE INVENTION

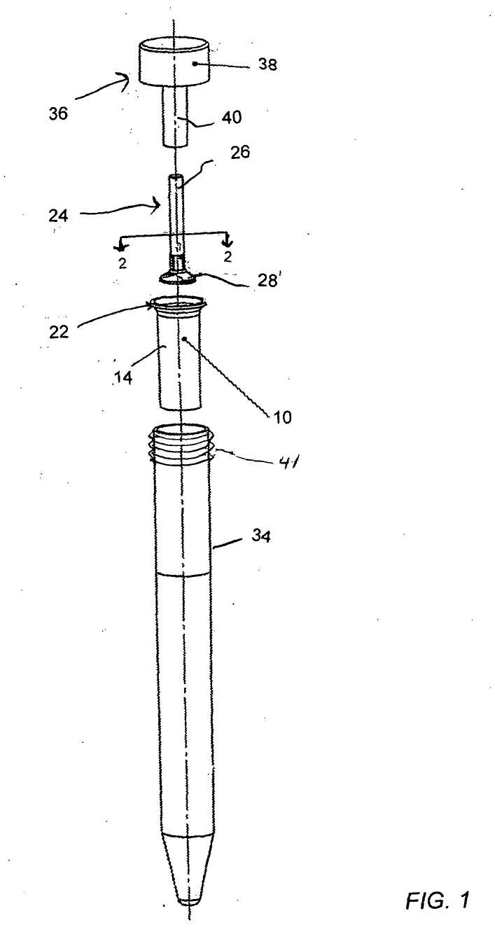

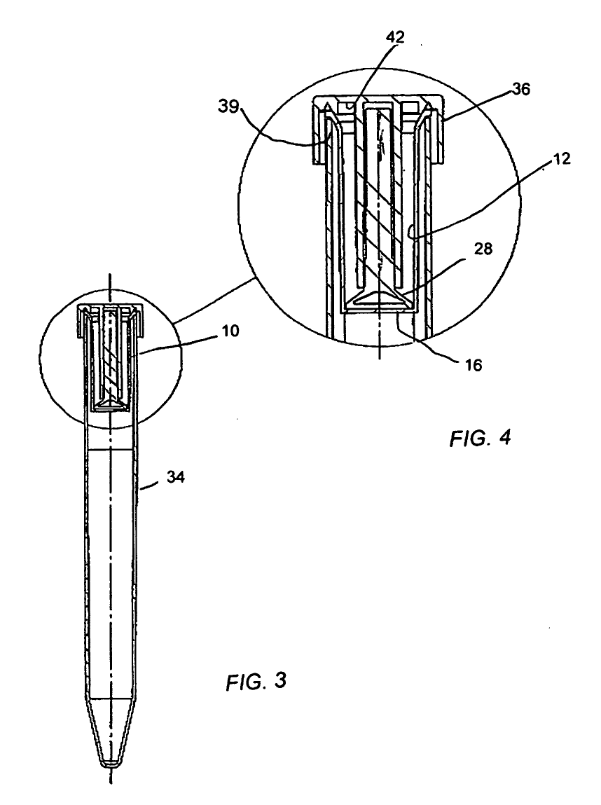

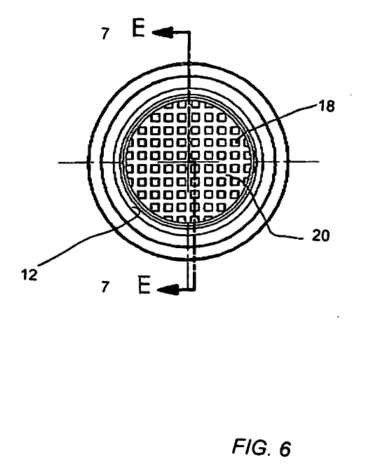

Referring to FIGS. 1-6, a sample of fecal matter is introduced into a tubular sample cup 10 formed of polypropylene and having inner and outer walls, respectively 12 and 14, and a bottom wall 16. The bottom wall 16 is formed with openings 18 defining a strainer 20 for the fecal sample, molded directly through the bottom wall16. The sample cup 10 has an open top end, indicated generally at 22, which will be described in more detail below.

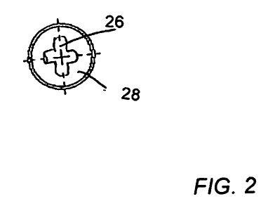

A plunger formed of polypropylene, indicated generally at 24, has a stem 26 terminating in flared bottom member 28 that is formed to close fit adjacent the inner wall 12 of the sample cup 10. Preferably, the outer dimension of the plunger bottom member 28 is coterminous with the inner surface 12 of the sample cup 10 so as to act as a piston in the sample cup whereby when the plunger 24 is progressively contacts the sample, the fecal matter contained in the cup 10 is extruded through the strainer 20. Referring more particularly to FIG. 2, the stem 26 can have a cruciate cross-section.

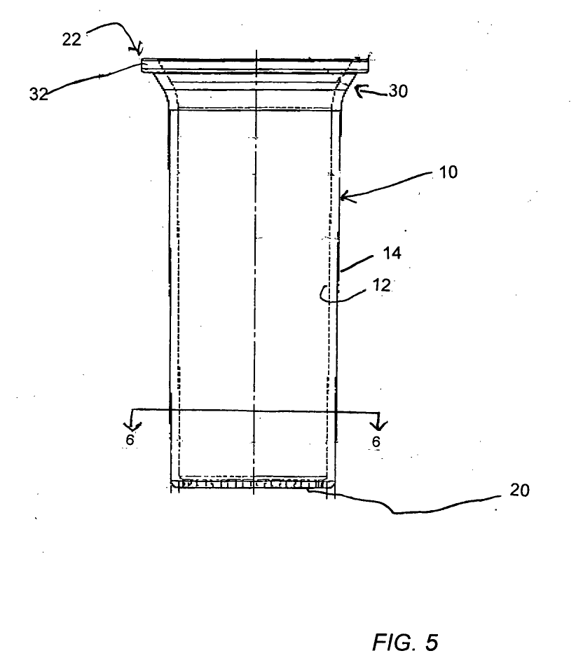

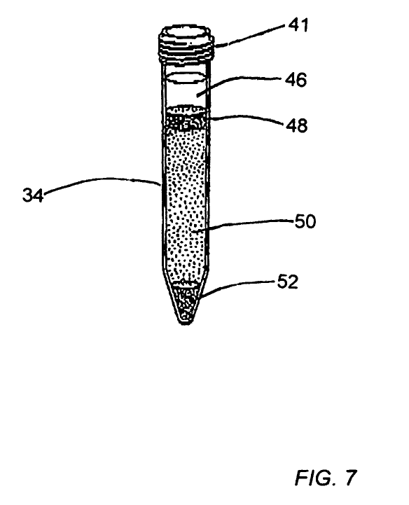

A screw cap pressure member 36 is provided formed of high density polyethylene in the shape of a hollow cap 38. A tubular member 40 axially depends from the bottom surface 42 of the cap 38. Referring more particularly to FIG. 5, the top end 22 of the sample cup 10 is formed with a flared edge region 30 extending outwardly from the outer wall of the sample cup, terminating in a top rim 32, which fits within the hollow cap 38 of the pressure member 36. Referring specifically to FIGS. 4 and 7, the inside surface of the hollow cap 38 is formed with a screw thread 39 that mates with a screw thread 41 on the top outer surface of the centrifuge tube 34.

In operation, a fecal sample is placed in the sample cup 10, which is already inserted into the open end 44 of a polypropylene centrifuge tube 34. The plunger 24 is then inserted into the sample cup 10 with the bottom member 28 of the plunger contacting the fecal matter. The pressure member 36 is disposed so that its tubular member 40 slides over the stem 26 of the plunger 24 until the bottom edge of the tubular member 40 contacts the upper surface of the flared bottom member 28 of the plunger 24. By screwing the cap 38 onto the open end of the centrifuge tube 34, the plunger further extrudes the fecal matter through the strainer 20 in the sample cup 10 and into the centrifuge tube 34 and the rim 32 of the sample cup 10 seals the open end of the centrifuge tube 34. The assembly of sample tube 10 and centrifuge tube 34 is then shaken horizontally and vertically to homogenize the mixture of feces material and solubilizer

A preservative, such as 10% buffered formalin or sodium acetate formalin or zinc PVA can be added to the centrifuge tube 34 before pushing plunger 24 into the sample cup 10. After shaking, the sample may be allowed to stand for 10 minutes or indefinitely to fixate the parasites. The sample cup 10 can then be removed and a surfactant such as Triton X-100 and ethyl acetate can be added. The nature of the solubilizer and cautions about its use and use of a surfactant are well known to the art, for example as set forth in the previously referred to Zierdt U.S. Pat. No. 4,081,356.

The cap 38 of the pressure assembly again screwed onto the open end of the centrifuge tube 34, following which the centrifuge tube is shaken and placed in a centrifuge. After centrifugation, four layers are present, namely, a top ethyl acetate layer 46, a fecal debris layer or plug 48 containing feces solids, a lower liquid layer of formalin 50, and a layer of packed sediment 52 in the bottom.

As described in the Zierdt patent, by first rimming the fecal plug with an applicator, holding the centrifuge tube 34 nearly horizontal, the ethyl acetate layer 46, the plug 48. and the formalin layer can be removed with an outward thrust, leaving the sediment 52 undisturbed. The walls of the centrifuge tube 34 can then be wiped with one or more loosely wrapped cotton swabs to remove adherent residual material so that the inner surface is cleaned to within one half inch of the sediment 52. Care should be taken not to hold the tube upright before the fecal solids and supernatant have been completely removed from the centrifuge tube 34 and the walls completely cleaned.

The centrifuge tube 34 is returned to an upright position and two to three drops of 10% formalin, or saline, are added, followed by thoroughly mixing the sediment. Slides are prepared with a transfer pipet, a coverslip is added and the sediment examined. Examination can be performed most accurately by methodically surveying the entire coverslip area with the 10× objective lens of the microscope.

1. An apparatus for recovering parasitic eggs and/or larvae from a fecal sample, comprising:
(a) a centrifuge tube having an open end on one side;
(b) a hollow sample cup for receiving a fecal sample, the sample cup having inner and outer walls and
(i) an outer dimension enabling it to fit within the open end of the centrifuge tube,
(ii) an open top end, and
(iii) a bottom end formed with a strainer for the fecal sample; and
(c) a plunger having a bottom end close fit and adjacent the inner wall of the sample cup to extrude the fecal sample through the strainer.

2. The apparatus of claim 1 in which the plunger has a stem extending normal to the bottom end of the plunger.

3. The apparatus of claim 2 including a press assembly for the plunger comprising:
(a) a tubular member fitting over the plunger stem and constructed so as to bear down on the bottom end of the plunger, and
(b) a thumb cap carried on the upper end of the tubular member.

4. The apparatus of claim 3 in which the tubular member of the press assembly is coterminous with the plunger stem at its bottommost position.

5. The apparatus of claim 3 in which the thumb cap is hollow and open at its bottom end and formed with a lid having upper and lower surfaces, the tubular member being axially secured normal to the lower surface of the lid, the upper surface of the lid having a size to accommodate a user's thumb.

6. The apparatus of claim 5 in which the top end of the sample cup is formed with a flared edge region sized to close fit inside the hollow cap.

7. The apparatus of claim 6 in which the flared edge region of the sample cup extends to close the centrifuge tube when the plunger extends fully into the sample cup.

8. The apparatus of claim 3 in which the thumb cap is formed with external screw threads and the open end of the centrifuge tube is formed with mating screw threads, to enable an assembly of the plunger, sample cup and centrifuge tube to be sealed together.

9. A method for concentrating parasitic eggs and/or larvae from a fecal sample, comprising extruding a fecal sample through a strainer into a centrifuge tube and centrifuging the extruded sample to concentrate parasite eggs and/or larvae at the bottom of the centrifuge tube.

10. The method of claim 9 in which fecal solubilizer is added to the fecal sample and/or centrifuge tube before, during and/or after extruding the sample.

11. The method of claim 9, in which the fecal sample is extruded by:
(a) placing the fecal sample in a sample cup fitted at its bottom end with a strainer and inserted into the end of the centrifuge tube;
(b) inserting a plunger into the sample cup, the plunger having a bottom end close fit to the inner wall of the sample cup;
(c) pushing the plunger into the sample cup to extrude the fecal sample through the strainer and into the centrifuge tube; and
(d) centrifuging the extruded sample to concentrate parasite eggs and/or larvae at the bottom of the centrifuge tube.

12. The method of claim 11 in which fecal solubilizer is added to the sample cup and/or centrifuge tube before, during and/or after pushing the plunger into the sample cup.
